(19) United States
(12) Patent Application Publication
(10) Pub. No.: US 2017/0370893 A1
(43) Pub. Date: Dec. 28, 2017

(54) UNIVERSAL MULTI-PARAMETER REMOTE WATER MONITORING SYSTEM

(71) Applicant: Smart Water Services, LLC, Decatur, GA (US)

(72) Inventor: Jonathan Paul West, Decatur, GA (US)

(21) Appl. No.: 15/635,781

(22) Filed: Jun. 28, 2017

Related U.S. Application Data
(60) Provisional application No. 62/355,361, filed on Jun. 28, 2016.

(51) Int. Cl. G01N 33/18; E03B 7/07; H04Q 9/00; E03B 9/04; H02S 99/00

(52) U.S. Cl. CPC G01N 33/1893; H04Q 9/00; E03B 9/04; E03B 7/072; E03B 7/075; H02S 99/00

(57) ABSTRACT

A remote hydraulic and water quality monitoring and telemetry device for being connected to a fire hydrant. The device connects to a hydrant nozzle via a swivel connection and includes a control valve mounted inside the nozzle to conserve space and release a determined volume of water from the hydrant lateral pipe to provide a sample from the main line pipe. The device includes a multi-parameter water quality sonde or sensors, data acquisition, and telemetry hardware. The door of the enclosure may include a solar panel. The device may rest on a plate mounted to the hydrant riser flange after the hydrant and upper hydrant valve stem are removed. The device may rest on the ground and be connected to a hydrant nozzle by a rotating pipe assembly which can be adjusted to be positioned at locations around the hydrant and at varying elevations relative to the hydrant nozzle.

### CROSS-REFERENCE TO RELATED APPLICATION

This application claims the benefit of priority of U.S. provisional patent application No. 62/355,361, titled “Universal Multi-Parameter Remote Water Monitoring System,” filed on Jun. 28, 2016, which is incorporated herein in its entirety by this reference.

### TECHNICAL FIELD

The presently disclosed subject matter is directed towards a multi-parameter, water monitoring, telemetry device, and more particularly a device that is installed at remote locations in water distribution piping networks for measuring hydraulic and water quality parameters.

### BACKGROUND

Public utility water systems are typically comprised of source water, treatment, conveyance, and distribution infrastructure including: raw water pumping stations, treatment facilities, high service pumping stations, booster pumping stations, elevated and ground storage tanks, re-pumping stations, control valve regulating station, and master meter stations between jurisdictions. Between these facilities is sometimes hundreds of miles of buried piping network making up the water distribution system. Typically, treatment facilities are highly regulated and include instrumentation for continuous monitoring of water levels, quality, and pressures. However, within the water distribution system, typically only water storage tanks, pumping stations, and master flow meters have instrumentation and remote monitoring through a supervisory control and data acquisition (SCADA) system. This leaves hundreds of miles of buried pipelines without any form of continuous remote monitoring.

Public water utilities are required by law to routinely collect water quality samples across the water distribution network. These typically take the form of grab samples which are analyzed on site or at the utility's laboratory. Currently, few water utilities monitor water quality continuously in the distribution network or they do but with a small number of remote sites. This is primarily due to the cost and complexity of integrating various instruments, providing adequate power and physical infrastructure, integrating with the existing SCADA network, operational complexity, and maintenance challenges.

Wireless communication has become ubiquitous in recent years due to the cellular consumer market and has led to the use of cellular communication as a low-cost alternative for telemetry communication for public water utilities. Advanced metering infrastructure (AMI) that use both licensed and unlicensed radio networks is also growing in adoption by public water utilities to wirelessly collect customer meter data, including consumption diurnal patterns, more frequently than traditional direct manual readings. Also, various forms of telemetry devices that monitor pressure, water quality, leak noise, level, and other parameters are being deployed in water distribution networks and communicating this data over cellular networks.

While continuous remote monitoring of hydraulic and water quality parameters in water distribution networks has been made easier with the wide spread deployment of cellular networks, there remain some physical constraints and challenges to monitoring water quality at remote locations which include: lack of access points to the pipeline at the desired monitoring point, cost of installing a buried vault at the desired location, lack of AC power, difficulty and cost of installing above ground solar panel in an urban roadway environment, and a requirement to provide an auxiliary sample stream to the water quality analyzer flow cell which cannot be wasted in the underground vault. This last requirement has led to the development of insertion type water quality probes that install directly in the pipe which can operate at high water pressures. However, some of these insertion type analyzers require sensor technologies that are not mature and may lack reliability and accuracy.

The common fire hydrant is primarily used for quick access to the pressurized public water system to fight fires and protect public health and property. Public water utilities also use fire hydrants for maintenance activities such as flushing to remove sediment and increase chlorine residual by evacuating older water where the chlorine has decayed below regulatory requirements or utility performance standards. Hydrants also provide a more readily available access point for water quality monitoring as they are typically above ground in many parts of the world solving the problem of analyzer flow-to-waste streams. However, one drawback of using hydrants as installation points for water quality monitoring systems is that the hydrant lateral pipe, which may be several meters long, has stagnant water which is not representative of water quality in the main line pipe. The water quality analyzer flow-to-waste stream in this case becomes a benefit for hydrant installation because the flow slowly removes the stagnant water from the hydrant lateral pipe, as opposed to a below ground vault installation where analyzer flow-to-waste is prohibitive. While water quality analyzers have different flow rate requirements that often range from 0.5 to 2.5 liters per minute and assuming an average hydrant pipe lateral length of 7 meters, the typical residence time of water in a hydrant lateral pipe can range from 1 to 8 hours. This leads to a significant delay of information for water utility operators who wish to make operational changes based on the telemetry data.

Accordingly, there remains a need for a device that avoids the various disadvantages and challenges associated with direct monitoring in water distribution network pipelines.

### SUMMARY

According to one aspect of the disclosed subject matter, a remote hydraulic and water quality monitoring and telemetry device configured for being connected to a common fire hydrant. The device includes a connection to one of the hydrant nozzles via a swivel connection. The device includes a vertical pipe connect by tee connection to the hydrant nozzle swivel connection. The vertical pipe contains a cylindrical flow cell that holds a multi-parameter water quality sonde. On top sits an angled enclosure which holds the data acquisition system and telemetry hardware. The door on the top of the angled enclosure includes a solar panel making the entire assembly resemble a common pole mounted solar panel system, which makes the installation low profile and less susceptible to potential vandals.

According to one aspect of the disclosed subject matter, a control valve assembly is located inside the hydrant in the throat of the larger pumper nozzle such that the valve is under hydraulic pressure both inside and outside the valve. The control valve is hydropneumatically actuated using external electronics in the external enclosure. The control valve is used to more quickly evacuate the stagnant water in the hydrant lateral pipe just before the data acquisition system collects a water quality data point ensuring the information is reflective of the water quality in the main line pipe. The control valve action reduces the stagnant water residence time to approximately 2 minutes as opposed to a 1-8 hour delay when using only a low flow water quality analyzer flow-to-waste stream.

According to one aspect of the disclosed subject matter, between the vertical pipe that houses the water quality sonde and the dry chamber that houses the electronics is an intermediate chamber with appurtenances including strainer and flow control valve to regulate the flow-to-waste stream through the water quality sonde flow cell and a DC latching solenoid valve that hydropneumatically controls the opening and closing of the larger internal hydrant control valve. The intermediate chamber is sealed from the vertical pipe and dry chamber to form a protective space and to prevent excessive moisture buildup that might harm sensitive electronics. The intermediate chamber and water quality sonde are accessed by way of a hinged panel inside the dry chamber.

According to one aspect of the disclosed subject matter, the data acquisition system housed in the upper angled enclosure is powered by a relatively large solar panel on the angled enclosure door in conjunction with a relative large battery bank in the same enclosure which provides the necessary power to facilitate frequent communication intervals and uploads to a central server and database. This frequent communication and data upload to a central server and database, along with the control valve effective sampling of the main line pipe more frequently, provides the water utility operator with up to the minute situational awareness of water distribution water quality and ability to respond more quickly to operational problems or emergency threats due to contamination.

According to one aspect of the disclosed subject matter, a multi-parameter water monitoring system is provided for being connected to a hydrant. The system may include an internal control valve mounted inside a fire hydrant nozzle, which can operate with water pressure internal and external to the valve body, and is hydraulically actuated using an electric controller to release water from inside to the outside of the hydrant.

The system may include a nozzle cap with at least one port that separates the internal control valve assembly from the rest of the water monitoring station device, wherein the ports are configured to sense pressure, provide continuous sample flow to a water quality sonde flow cell, provide hydropneumatic control of the internal hydrant control valve, or evacuate water from the hydrant body, riser pipe, and lateral pipe. The system may include a low profile mechanical assembly which houses all of the necessary components for a remote water monitoring and telemetry station which resembles a pole mounted solar panel in order to minimize vandalism. The system may include a low profile mechanical assembly that connects to a typical fire hydrant nozzle, with short pipe connected to a vertical pipe which houses a vertical flow cell and water quality sonde or multiple sensors.

The system may include a low profile mechanical assembly defining at least one intermediate compartment for mounting a sample flow regulator which provides both a low continuous flow to the water quality sonde and an intermittent flow from the internal control valve, and providing a barrier space between the open atmospheric lower vertical pipe and the upper dry electrical compartment housing the data acquisition system and power supply. The system may include an assembly including an internal subpanel below an exterior enclosure door that provides access to all system components and for removing the water quality sondes for maintenance. The system may include a harness assembly for securing a water monitoring station to a hydrant at two or more points so as not to require support from the ground, including at least one point being a hydrant flow nozzle and one point being the hydrant body.

The system may include an assembly including an inner and outer recirculation sampling tube and circulation pump where the outer recirculation tube can be installed through the open hydrant nozzle, through the hydrant body, riser pipe, open primary hydrant valve, and partially through a hydrant lateral pipe to a closed secondary valve, and is connected to the interior of a hydrant nozzle cap/port, where after the inner recirculation tube can be inserted through a compression seal through the nozzle cap/port into the outer recirculation tube, where after the secondary lateral valve is opened and pressurizes the entire hydrant assembly and recirculation tubes, where after the inner recirculation tube is pushed until the end reaches the end of the hydrant lateral pipe and cross section of the main line pipe forming an internal recirculation system so that water can be conveyed using an electric pump from the main line pipe through the inner recirculation tube, through the water quality sonde flow cell and back into the hydrant assembly where water is returned to the water distribution network without wasting water externally to the hydrant and assembly.

According to one aspect of the disclosed subject matter, a software program is provided that controls the open and close times of a hydrant control valve for rapid sampling of water quality from the main line piping. The software may include or utilize user adjustable settings in communication with a controller software via local configuration software or remote web-based software over wireless IP connection. The software may include or utilize logic that removes a determined volume of stagnant water in the hydrant lateral pipe, vertical riser, and hydrant body based on user entered values of pipe diameter, length, and hydrant control valve flow rate. The software may include or utilize logic that removes a determined volume of stagnant water from the hydrant lateral pipe, vertical riser, and hydrant body just prior to the data acquisition system scanning the water quality sonde.

According to one aspect of the disclosed subject matter, a lower valve stem mechanical lock assembly is provided for dry barrel hydrants that holds the lower hydrant stem in a vertical and horizontal position at the internal break away coupling point, with the upper hydrant stem removed, such that the permanent hydrant valve in the shoe remains open and covering the below ground weep holes, allowing a hydrant riser pipe to be pressurized up to an installed cap located where the above-ground hydrant would normally be connected. A stem cap may be provided that covers the top of the lower hydrant operating stem and holds the valve stem in a locked horizontal and vertical position. An isolation plate may be provided that takes the place of the hydrant body and seals the hydrant below ground riser pipe allowing it to be pressurized from the underground water piping network. An isolation plate may be provided that contains ports for connecting to a water quality monitoring station. The stem cap may be excluded as not required because of the lack of below ground hydrant valve or valve stem.

According to one aspect of the disclosed subject matter, a method may be provided that includes: using a control valve positioned within a hydrant; allowing periodic release of water flow to one or more measurement analyzers for measuring one or more characteristics of the released water flow; and transmitting the measured values to a remote entity. The one or more measurement analyzers may include a first sonde positioned in series upstream of a second sonde. The released water flow may be discharged to atmosphere.

According to one aspect of the disclosed subject matter an apparatus is provided that may include a hydrant valve and a flow control valve positioned in a throat of the hydrant nozzle for periodically releasing water. The apparatus may further include a circulation chamber external to the hydrant valve and in fluid communication with the flow control valve. The apparatus may further include one or more measurement analyzers within the circulation chamber for measuring one or more characteristics of the released water. The apparatus may further include a transmission system for transmitting the measured characteristics.

According to one aspect of the disclosed subject matter, a method includes: using a rotating supply pipe assembly to connect a hydrant nozzle to a water monitoring and control valve station, allowing the station to be installed at various locations around the hydrant to avoid physical obstacles, on the ground regardless of the distance between the hydrant nozzle and the ground, which can vary significantly depending on the burial depth of the hydrant, and at various ground inclines.

According to one aspect of the disclosed subject matter, an apparatus includes: a hydrant swivel adapter nozzle connection; a segment of pipe; a coupling; a flexible hose conduit; a prefabricated enclosure pad that rests on the ground and provides a pathway for one or more flexible conduits; an enclosure housing that rests on the equipment pad; a control valve within the enclosure housing connecting to the flexible hose conduits; one or more measurement analyzers within the enclosure housing for measuring one or more characteristics of the released water from a control valve; a discharge pipe that exits the enclosure and discharges to atmosphere; and a transmission system for transmitting the measured characteristics.

According to one aspect of the disclosed subject matter, a system and corresponding method for measuring water quality are provided. The system includes: a control valve for positioning internal within a hydrant, and allowing a release of water flow; at least one analyzer for determining at least one measured value for at least one characteristic of the water flow; and a transmitter for transmitting the measured value to a remote entity.

A controller may be coupled to the control valve to cause the control valve to release water periodically. A controller may be coupled to the control valve to cause the control valve to release a volume of water determined to empty a hydrant lateral to get a water-quality sample from a mainline. An enclosure may be provided for mounting on a hydrant, wherein the enclosure houses at least one water analyzer. One water analyzer may be mounted within the hydrant.

The released water flow may be discharged outside the hydrant. The control valve may be located in a throat of a pumper nozzle of the hydrant. The flow control valve may be connected to a cap held in place by a nozzle swivel adapter that seals the pumper nozzle when the hydrant is pressurized with water.

A solar collector may provide power for at least one of the control valve and analyzer. A battery may provide power for at least one of the control valve and analyzer.

The summary is provided to introduce a selection of concepts in a simplified form that are further described below in the detailed description. This summary is not intended to identify key features or essential features of the claimed subject matter, nor is it intended to be used to limit the scope of the claimed subject matter. Further, the claimed subject matter is not limited to implementations that solve any or all disadvantages noted in any part of the disclosure.

### BRIEF DESCRIPTION OF THE DRAWINGS

The foregoing summary, as well as the following detailed descriptions of preferred embodiments, is better understood when read in conjunction with the appended drawings. For the purposes of illustration, there is shown in the drawings exemplary embodiments; however, the presently disclosed subject matter is not limited to the specific methods and instrumentalities disclosed. In the drawings:

FIG. 1 is an elevation view, partially in cross section, of a typical hydrant installation including: hydrant body, riser pipe, hydrant valve, lateral pipe, isolation valve, and main line pipe.

FIG. 2 is a block diagram illustrating the interaction of the major system components of the apparatus of the present disclosure.

FIG. 3 is an elevation front view illustrating the major external system components of the apparatus of the present disclosure

FIG. 4 is an elevation side view illustrating the major external system components of the apparatus of the present disclosure.

FIG. 5 is an isometric view of an apparatus in accordance with the present disclosure.

FIG. 6 is a schematic diagram illustrating the wireless communication pathway from the apparatus of the present disclosure to remote server, database, and human machine interface (HMI) display.

FIG. 7 is a schematic diagram illustrating the hydrant control valve program logic of the apparatus of the present disclosure.

FIG. 8 is an elevation view illustrating a hydrant riser pipe isolation plate and lower valve stem lock, of an apparatus in accordance with the present disclosure, used for connecting the apparatus of the present disclosure directly to the top of the hydrant riser with the upper hydrant body removed.

FIG. 9 is an elevation view, partially in cross section, of an apparatus in accordance with the present disclosure, including a typical hydrant installation and internal recirculation sampling line and pump.

FIG. 10 is an elevation view, partially in cross section, of an apparatus in accordance with the present disclosure, including a typical hydrant installation, a monitoring and control valve station located on an equipment pad on the ground and connected by a rotating pipe assembly.

FIG. 11 is an elevation view, partially in cross section, of an apparatus in accordance with the present disclosure, including a typical hydrant at various heights above ground and a rotating pipe assembly connecting the hydrant nozzle to the base entry point of the monitoring and control valve station.

FIG. 12 is a plan view of an apparatus in accordance with the present disclosure, including a typical hydrant installation next to vegetation and man-made ground features, showing multiple installation location options for the water monitoring and control valve station.

### DETAILED DESCRIPTIONS

The presently disclosed subject matter now will be described more fully hereinafter with reference to the accompanying drawings, in which some, but not all embodiments are shown. Indeed, this disclosure may be embodied in many different forms and should not be construed as limited to the embodiments set forth herein; rather, these embodiments are provided so that this disclosure will satisfy applicable legal requirements. Like numbers refer to like elements throughout.

When elements are referred to as being “connected” or “coupled”, the elements can be directly connected or coupled together, or one or more intervening elements may also be present. In contrast, when elements are referred to as being “directly connected” or “directly coupled,” there are no intervening elements present.

The subject matter may be embodied as devices, systems, methods, and/or computer program products. Accordingly, some or all of the subject matter may be embodied in hardware and/or in software (including firmware, resident software, micro-code, state machines, gate arrays, etc.) Furthermore, the subject matter may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be for example, but not limited to, an electronic, magnetic, optical, electromagnetic, infrared, or semiconductor system, apparatus, device, or propagation medium. By way of example, and not limitation, computer-readable media may comprise computer storage media and communication media.

Computer storage media is non-transitory and includes volatile and nonvolatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage components, or any other medium which can be used to store the desired information and may be accessed by an instruction execution system. Note that the computer-usable or computer-readable medium can be paper or other suitable medium upon which the program is printed, as the program can be electronically captured via, for instance, optical scanning of the paper or other suitable medium, then compiled, interpreted or otherwise processed in a suitable manner if necessary, and then stored in computer memory.

Communication media typically embodies computer-readable instructions, data structures, program modules or other data in a modulated data signal such as a carrier wave or other transport mechanism and includes any information delivery media. The term “modulated data signal” can be defined as a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above-mentioned should also be included within the scope of computer-readable media.

When the subject matter is embodied in the general context of computer-executable instructions, the embodiment may comprise program modules, executed by one or more systems, computers, or other devices. Generally, program modules include routines, programs, objects, components, and data structures (and the like) that perform particular tasks or implement particular abstract data types. Typically, the functionality of the program modules may be combined or distributed as desired in various embodiments.

Recitation of ranges of values herein are merely intended to serve as a shorthand method of referring individually to each separate value falling within the range, unless otherwise indicated herein; and each separate value is incorporated into the specification as if it were individually recited herein. Therefore, any given numerical range shall include whole and fractions of numbers within the range. For example, the range “1 to 10” shall be interpreted to specifically include whole numbers between 1 and 10 (e.g., 1, 2, 3, . . . 9) and non-whole numbers (e.g., 1.1, 1.2, . . . 1.9).

Although process (or method) steps may be described or claimed in a particular sequential order, such processes may be configured to work in different orders. In other words, any sequence or order of steps that may be explicitly described or claimed does not necessarily indicate a requirement that the steps be performed in that order unless specifically indicated. Further, some steps may be performed simultaneously despite being described or implied as occurring non-simultaneously (e.g., because one step is described after the other step) unless specifically indicated. Where a process is described in an embodiment, the process may operate without any user intervention.

For the purposes of promoting an understanding of the principles of the present disclosure, reference will now be made to preferred embodiments, and specific language will be used to describe the same. It will nevertheless be understood that no limitation of the scope of the disclosure is thereby intended; such alteration and further modifications of the disclosure, as illustrated herein, is being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

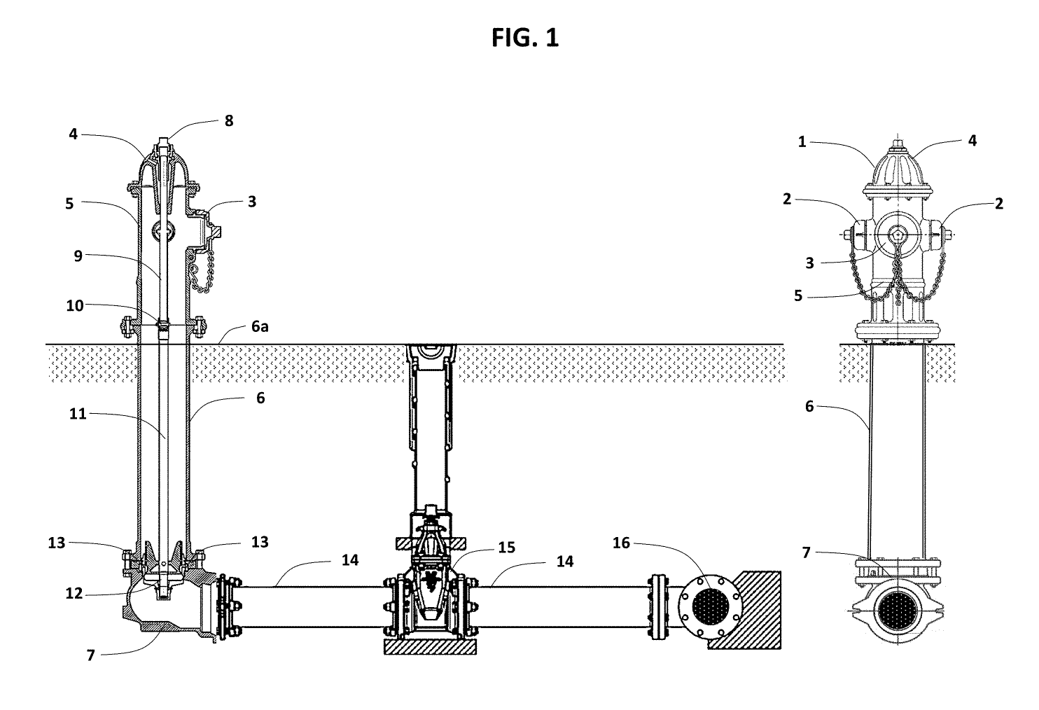

As shown in FIG. 1 a typical fire hydrant 1 consists of two (2) 2½″ nozzles 2 and a single 4.5″ pumper nozzle 3. The hydrant bonnet 4 sits on top of the main hydrant body 5 which is connected to a riser pipe 6 which is connected to the hydrant shoe 7. The ground is illustrated as 6a. The bonnet 4 holds the valve operating nut 8 and upper valve stem 9 which is connected to the lower valve stem 11 using a break away coupling 10. The lower valve stem 11 is connected to the hydrant valve 12. When in the closed upward position, one or two weep holes 13 in the shoe 7 are uncovered, thereby allowing water to drain from the hydrant 1 and riser pipe 6 into the ground. When the hydrant valve 12 is in the open or downward position, the hydrant valve 12 covers the weep holes 13 in order to prevent pressurized water from escaping. The hydrant secondary valve 15 is typically open, thereby allowing the hydrant lateral pipe 14 to be fully pressurized from the main line pipe 16.

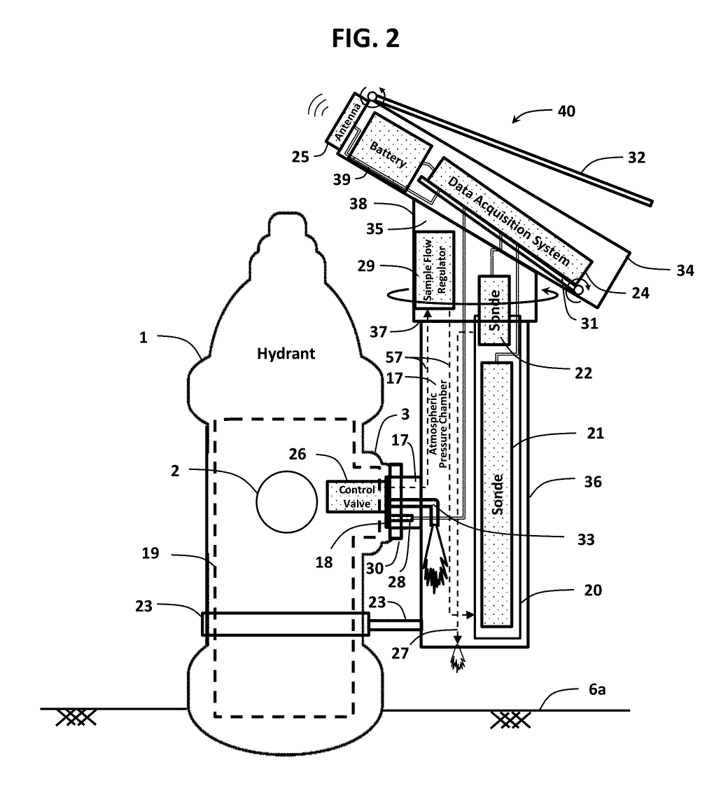

As shown in FIG. 2, a block diagram elevation view illustrates a water quality monitoring station 40 where a flow control valve 26 is located inside the hydrant in the throat of the pumper nozzle 3. An interior void 19 of the hydrant is illustrated. The flow control valve 26 is connected to a cylindrical cap/port 18 which is held in place by a nozzle swivel adapter 30 to seal the pumper nozzle 3 when the hydrant is pressurized with water.

Connected to the swivel adapter 30 is the bottom cylindrical enclosure 36, including fabricated tee, which supports an upper enclosure 38. The upper enclosure includes two compartments, the intermediate wet panel enclosure 35 and dry electrical compartment 34. The upper enclosure 38 swivels on a rotating flange 37 to orient the solar panel 32 toward the sun. The flow control valve 26 is hydropneumatically controlled via sensing lines that connect to a sample flow regulator 29, located in the intermediate wet panel compartment 35, which is controlled by electronic solenoid. The sample flow regulator 29 provides a constant flow to the low water pressure chamber/flow cell 20 in which the primary multi-parameter water quality sonde 21 is located. A secondary sonde 22 can be located at the top of the flow cell 20. The primary sonde 21 and secondary sonde 22 are connected to the central data acquisition system 24 located in the dry electrical compartment 34 and is mounted on a hinged electrical subpanel that opens to allow the sondes 21 22 to be removed from the flow cell 20 and to adjust the sample flow regulator 29. Likewise, the solar panel/hinged door 32 is opened to access the data acquisition system 24 and batteries 39. A harness 23 secures the water quality monitoring station 40 to the hydrant 1.

In operation, water flows from the hydrant 1 into atmospheric void 17 through sample tubing 57 into the sample flow regulator 29. The regulated water then flows through sample tubing 57 and through the sonde flow cell 20 before being discharged at discharge outlet 27.

The sonde flow cell 20 in at least one embodiment is an instrument probe that transmits data, for example to a remote device or entity. For example, the flow control valve 26 positioned internal within the hydrant 1 can allow a release of water flow and the sonde flow cell 20 can include at least one analyzer that determines a measured value for at least one characteristic of the water flow. The measured value can be transmitted to a separate nearby or remote entity or device. For example, the transceiver 25 in FIG. 2, which functions as both a transmitter and a receiver in at least one embodiment, has an antenna that can be used to transmit the measured value and receive signals.

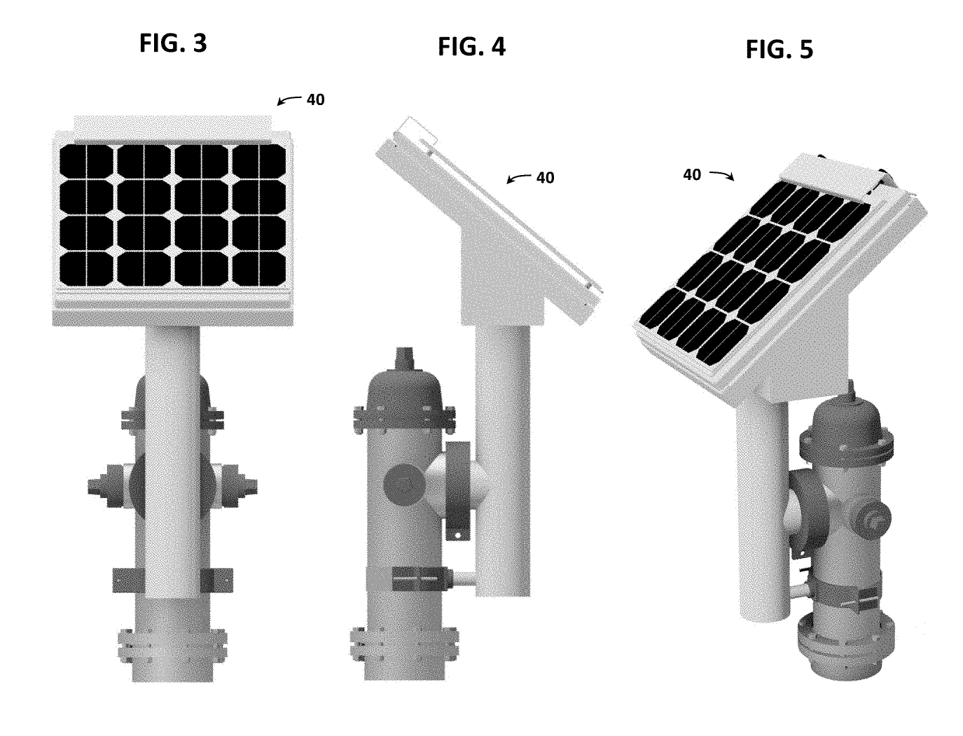

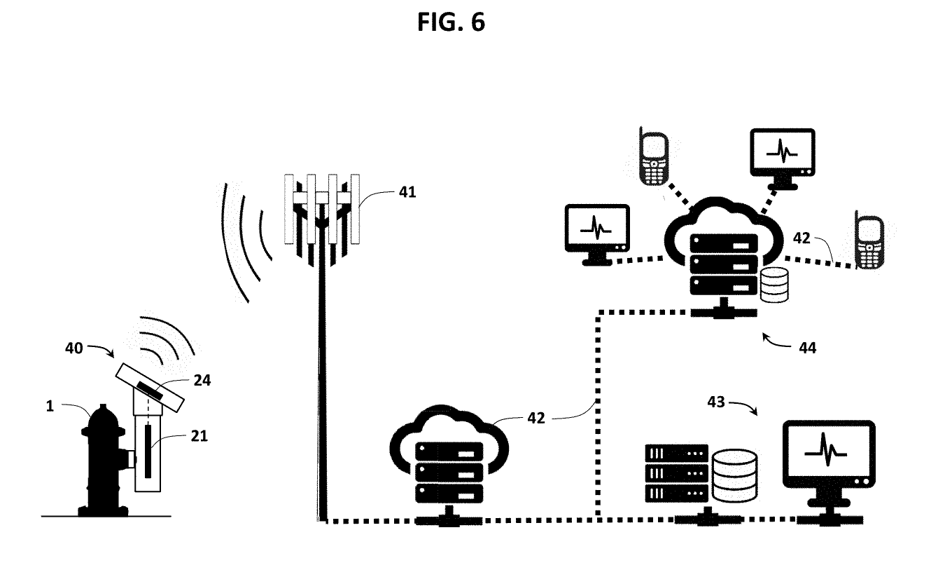

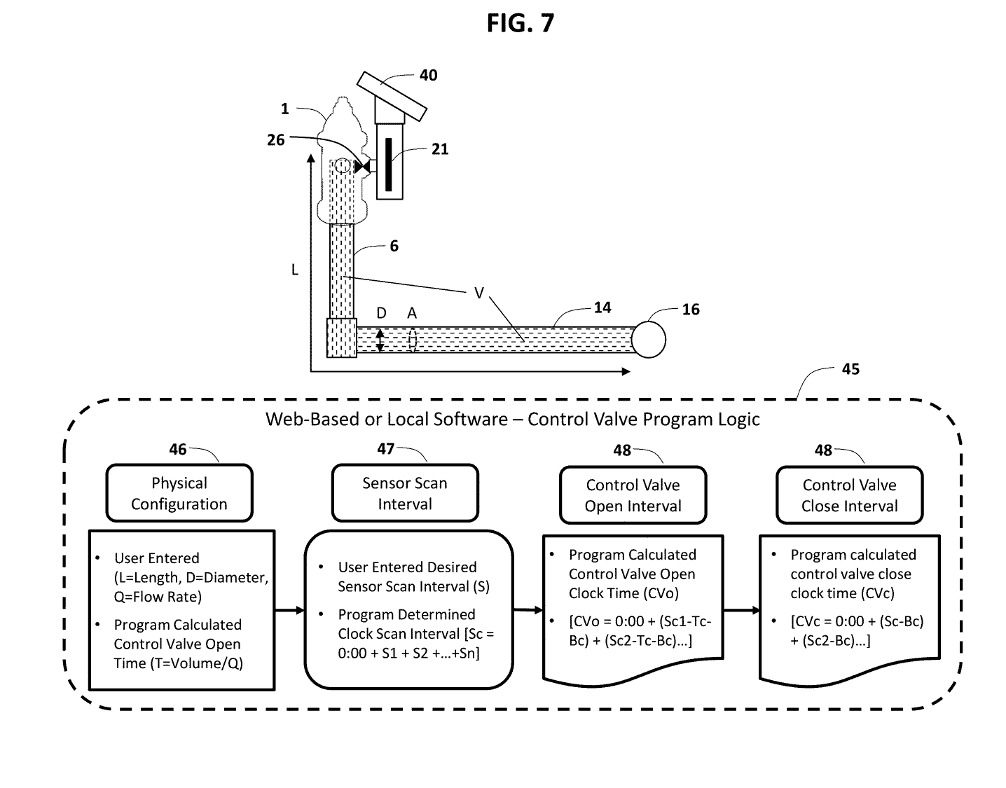

As shown in FIGS. 3, 4, and 5, a front, side, and isometric elevation view illustrates a water quality monitoring station 40 connected to a typical fire hydrant.

As shown in FIG. 6, a schematic diagram illustrates one embodiment of the disclosure describing the water quality sonde 21, data acquisition and local data storage device 24, wireless communication to a base station or cell tower 41 and communication network 42, to a local area network (LAN) 43 including communication server, database storage, and human machine interface (HMI), or to a managed cloud computing environment 44 including web-based HMI.

As shown in FIG. 7 a schematic diagram illustrates one embodiment describing the control valve sampling program logic 45, which can be user adjusted in a local configuration software or remotely from a web-based HMI, to evacuate the stagnant water in the hydrant lateral 14 and riser pipe 6 prior to scanning the water quality sonde 21. Physical parameters 46 are user entered (L=Length of Hydrant Lateral Plus Riser, D=Hydrant Lateral/Riser Internal Diameter, and Q=Actual Control Valve Flow Rate). The program calculates the volume of water (V) and determines the time (T) it takes to evacuate the water in the hydrant lateral 14, riser pipe 6, and hydrant body 1. The user enters the desired water quality sensor scan interval 47 (S), and the program calculates the sensor scan clock times based on the equation Sc=0:00+S1/(24×60)+S2/(24×60)+ . . . +Sn/(24×60)) where S>T+B and where B is a user entered buffer time after the control valve closes and the water quality sensor is scanned by the program. The program then determines the control valve open and close clock time 48 based on the equation CVo=[0:00+(S−T−B)/(24×60)+ . . . +(Sn−T−B)/(24×60)] and CVc=[0:00+(S−B)/(24×60))+ . . . +(Sn−B)/(24×60)] which evacuates a volume of water (V) from the hydrant lateral 16, riser pipe 6, and hydrant body 1 prior to the sensor scan interval by an amount of time B. The sampling control valve program logic 45 provides a sample from the main line pipe 16 in the city street to the water quality sonde 21 in a relatively short time frame (a few minutes as opposed to several hours) providing the water utility operator with up to the minute situational awareness of water distribution network water quality and ability to respond more quickly to operational problems or emergency threats due to contamination.

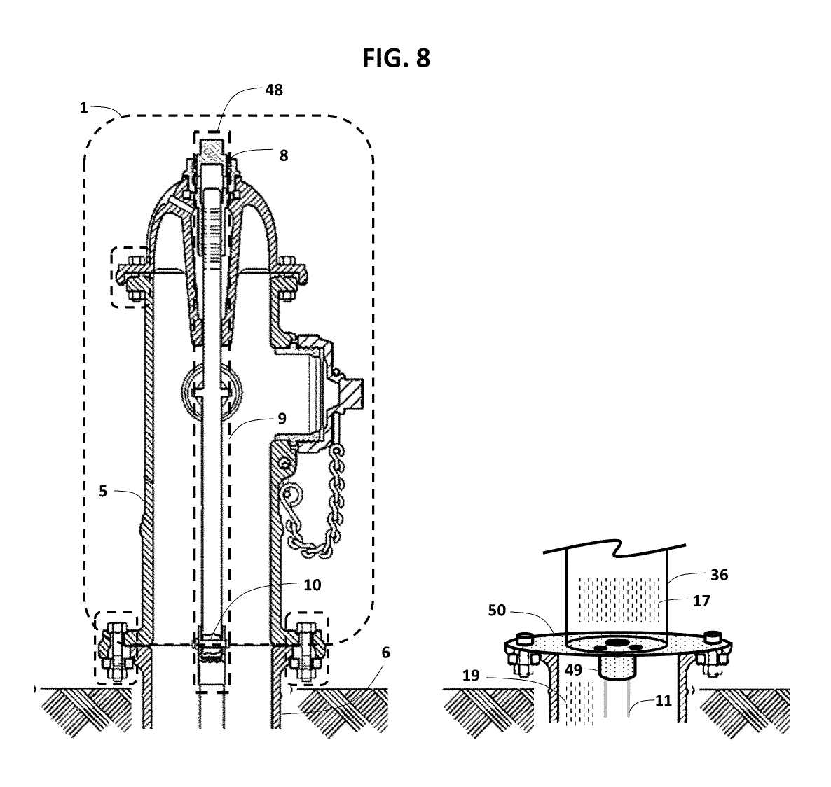

As shown in FIG. 8, the water quality monitoring station can be connected directly to the top of the hydrant riser 6 with the upper hydrant body 1 removed. For dry barrel hydrants, the upper valve stem assembly 48, consisting of the hydrant nut 8, upper valve stem 9, and breakaway coupling 10, is removed along with the upper hydrant body 1. A lower valve stem locking assembly 49 is connected to the top of the remaining lower stem 11 and connected to the riser isolation plate 50, positioning the lower valve stem and hydrant valve 12 in the open position which closes the weep holes 13 in the hydrant shoe 7, enabling the hydrant riser pipe 6 of a dry barrel hydrant to be pressurized such that a water quality monitoring station can be connected to the top of the riser pipe 6 using a flange or coupling connection.

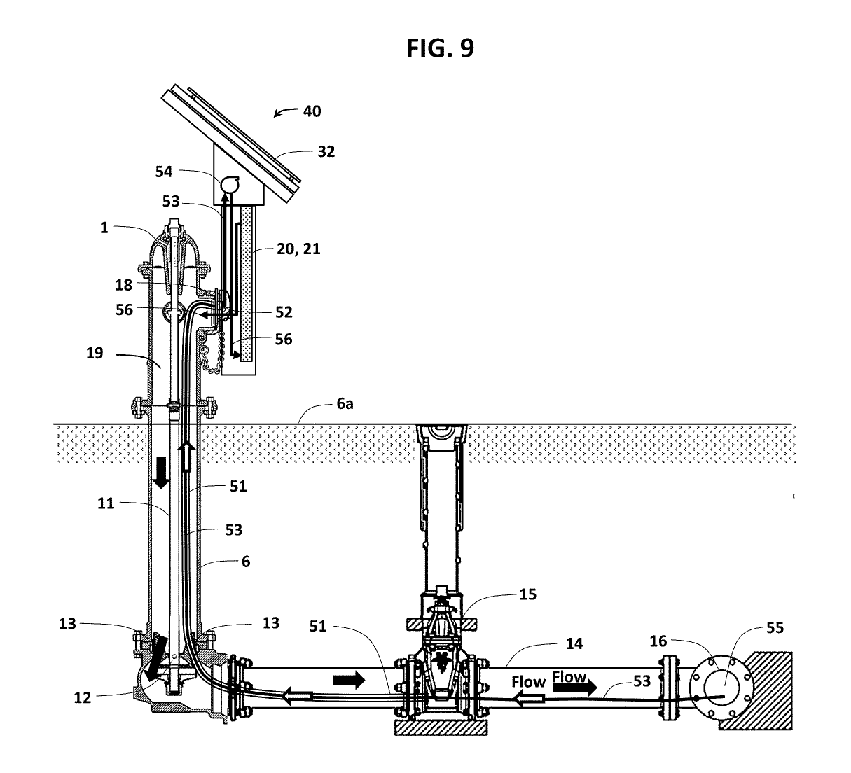

As shown in FIG. 9, the water quality monitoring station 40 can be configured with a sleeve tube 51, inner recirculation sampling tube 53, and circulation sampling pump 54 to supply the water quality sonde flow cell 20 and keep water contained within the water distribution piping network. The outer sampling tube 51 is installed after the hydrant lateral secondary valve 15 is closed and the hydrant primary valve 12 is open. The outer sampling tube 51 is run through an open hydrant pumper nozzle 3 into the hydrant interior void 19 through the open hydrant primary valve 12, and to the closed secondary lateral valve 15, and then connected to the pumper nozzle cap/port 18 which is connected securely to the pumper nozzle 3. The smaller inner sample tube 53 is inserted into the larger outer sampling tube 51 and run to the closed secondary lateral valve 15. The compression seal 52 on the nozzle cap/port 18 is tightened and the secondary lateral valve 15 is opened allowing the hydrant riser pipe 6 and hydrant body 1 to be pressurized from the water distribution network. The inner sampling tube 53 is then pushed further so that the end reaches the main line pipe 16. The circulation sampling pump 54 is supplied water from the inner recirculation tube 53 and pumps it through high pressure tubing 56 through the water quality sonde flow cell 20 and back to the pumper nozzle cap/port 18 so that return water is pumped back into the hydrant interior void 19, down the hydrant riser 6, along the hydrant lateral 14, and back to the main line pipe 16 which forms a mixing zone 55 at the intersection of the hydrant lateral pipe 14 and the main line pipe 16.

While the embodiments have been described in connection with the preferred embodiments of the various figures, it is to be understood that other similar embodiments may be used or modifications and additions may be made to the described embodiments for performing the same function without deviating therefrom. Therefore, the disclosed embodiments should not be limited to any single embodiment, but rather should be construed in breadth and scope in accordance with the appended claims.

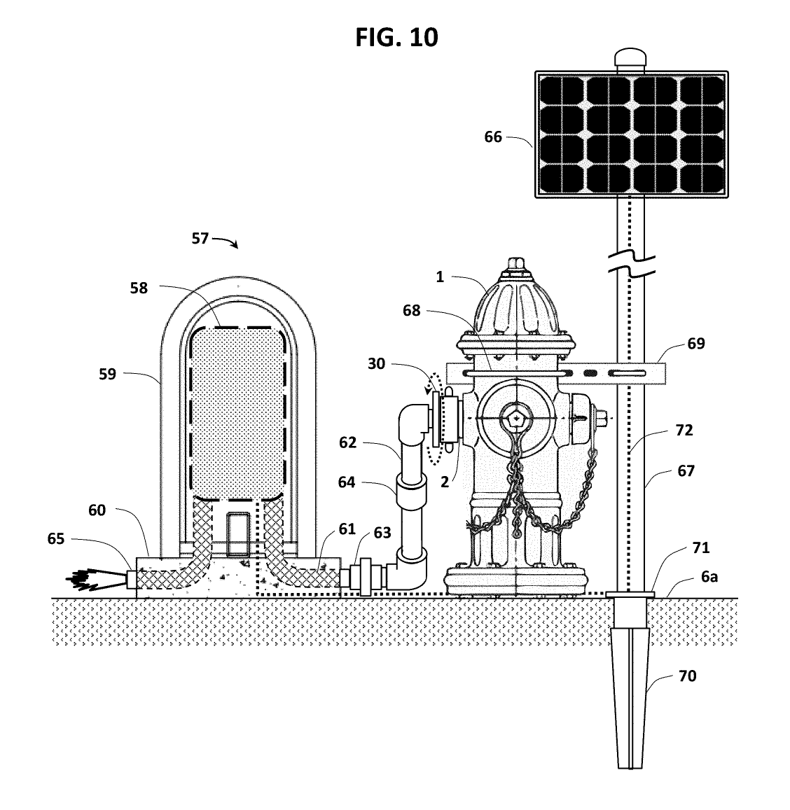

As shown in FIG. 10, a similar embodiment consists of a ground-based water monitoring station 57, which contains water monitoring, control, and communication equipment 58 and is housed in a secure enclosure 59 which is mounted on a pre-fabricated equipment pad 60, which can be mounted on the ground 6a near the hydrant 1. The pre-fabricated equipment pad 60 contains a high pressure rated flexible hose 61 which allows the water monitoring station 57 to be installed on inclined ground surfaces which are out of plane with the hydrant nozzle connection 2. The water quality monitoring station 57 is supplied with water through a rotating pipe assembly 62 which is connected to the flexible hose 61 using a standard pipe coupling 63 and to the hydrant nozzle 2 using a hydrant nozzle swivel adapter 30. The rotating pipe assembly 62 may also have a flow meter 64 to monitor the volume of water expelled from the discharge pipe 65. The water monitoring station 57 may be powered by a solar panel 66 which is mounted to a pole 67 which is secured to the hydrant 1 in two places, on the hydrant using a large U-bolt 68 and channel strut assembly 69 and to the ground 6a using an embedded soil anchor 70 or surface mount anchor plate 71. The solar panel 66 is wired to the water quality monitoring, control, and communication equipment 58 through an electrical conduit 72.

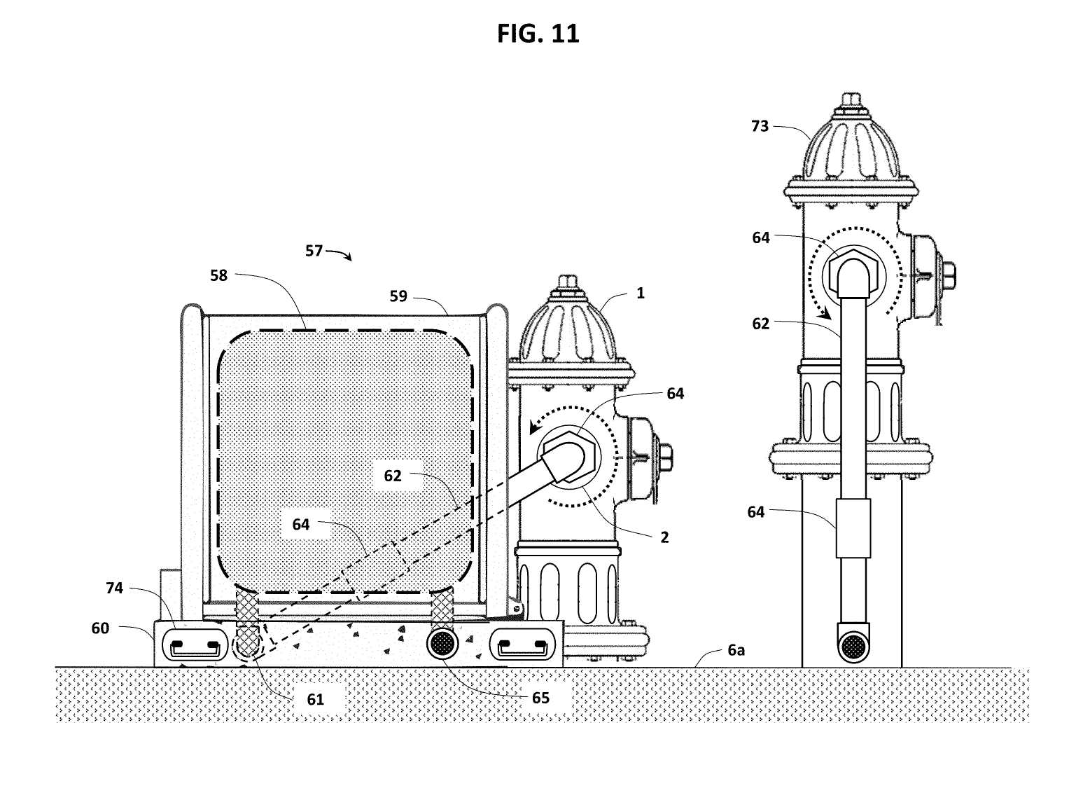

As shown in FIG. 11, the water monitoring station 57 is shown with a standard hydrant 1 mounted in the ground at proper height and an abnormal hydrant installation 73 which either extends out of the ground and or is partially buried. Prior to tightening the nozzle swivel adapter 30, the rotating pipe assembly 62 is positioned to match the elevation of the flexible hose 61 connection in the pre-fabricated base pad 60 which is either at an elevation below, equal, or above the hydrant nozzle 2. Handles 74 are mounted to the pre-fabricated base pad 60 so that the water monitoring station 57 can be installed or removed from service quickly and easily transported to another site.

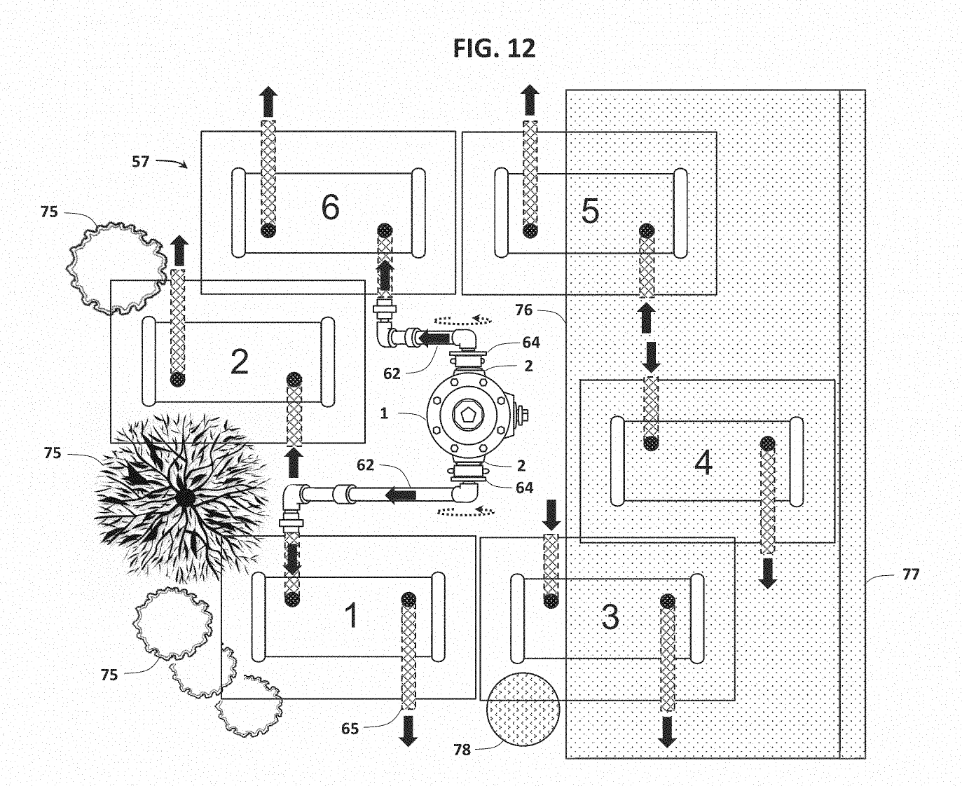

As shown in FIG. 12, a plan view illustrates that the water monitoring station 57 can be installed at alternate locations (1, 2, 3, 4, 5, or 6) around the hydrant 1 to avoid physical obstructions which may include vegetation 75 or manmade structures such as a sidewalk 76, street curb 77, or utility pole 78. The rotating pipe assembly 62 can be installed on either hydrant nozzle 2 and rotated to an ideal location and elevation such as location #6.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

What is claimed is:

1. A method of measuring water quality, the method comprising:
using a control valve positioned internally within a hydrant, allowing a release of water flow;
determining at least one measured value for at least one characteristic of the water flow using at least one analyzer; and
transmitting the measured value to a remote entity.

2. The method according to claim 1, wherein allowing release of water flow comprises periodically releasing water.

3. The method according to claim 1, wherein allowing release of water flow comprises releasing a volume of water determined to empty a hydrant lateral pipe and riser to get a water-quality sample from a mainline pipe.

4. The method according to claim 1, wherein at least one analyzer sensor is housed by an enclosure mounted on the hydrant.

5. The method according to claim 1, wherein at least one analyzer sensor is mounted within the hydrant nozzle.

6. The method according to claim 1, wherein allowing release of water flow comprises discharging the water flow outside the hydrant.

7. The method according to claim 1, wherein the control valve is located in a throat of a hydrant nozzle.

8. The method according to claim 1, wherein the flow control valve is connected to a cylindrical cap that is held in place by a swivel adapter that seals the hydrant nozzle when the hydrant is pressurized with water.

9. The method according to claim 1, wherein a solar collector provides power for at least one of the control valve, analyzer, and telemetry.

10. The method according to claim 1, wherein a battery provides power for at least one of the control valve and analyzer.

11. A system for measuring water quality, the system comprising:
a control valve for positioning internal within a hydrant, and allowing a release of water flow;
at least one analyzer for determining at least one measured value for at least one characteristic of the water flow; and
a transmitter for transmitting the measured value to a remote entity.

12. The system according to claim 11, further comprising a controller coupled to the control valve to cause the control valve to release water periodically.

13. The system according to claim 11, further comprising a controller coupled to the control valve to cause the control valve to release a equivalent volume of water determined to empty a hydrant lateral pipe to get a water-quality sample from a mainline pipe.

14. The system according to claim 11, further comprising an enclosure for mounting on a hydrant, wherein the enclosure houses the at least on analyzer sensor.

15. The system according to claim 11, wherein the at least one analyzer sensor is mounted within the hydrant.

16. The system according to claim 11, wherein allowing release of water flow comprises discharging the water flow outside the hydrant.

17. The system according to claim 11, wherein the control valve is located in a throat of a hydrant nozzle.

18. The system according to claim 11, wherein the flow control valve is connected to a cylindrical cap held in place by a nozzle swivel adapter that seals the nozzle when the hydrant is pressurized with water.

19. The system according to claim 11, further comprising a solar collector for providing power for at least one of the control valve and analyzer.

20. The system according to claim 11, further comprising a battery provides power for at least one of the control valve and analyzer.

21. A method of measuring water quality, the method comprising:
using a rotating pipe assembly to connect a hydrant nozzle, at various heights above ground, to a water monitoring and control valve station, allowing the station to be installed at various locations around the hydrant to avoid physical obstacles, and on the ground regardless of the distance and angle between the hydrant nozzle and the ground.

22. A system for measuring water quality, the system comprising:
a hydrant swivel adapter nozzle connection;
a rotating pipe;
a coupling;
a flexible hose conduit;
a prefabricated enclosure pad that rests on the ground and provides a pathway for one or more flexible conduits;
an enclosure housing that rests on the equipment pad;
a control valve within the enclosure housing connecting to the flexible hose conduits;
one or more measurement analyzers within the enclosure housing for measuring one or more characteristics of the released water from a control valve;
a discharge pipe that exits the enclosure and discharges to atmosphere; and
a transmission system for transmitting the measured characteristics.
